(19) United States
(12) Patent Application Publication
(10) Pub. No.: US 2004/0101478 A1
(43) Pub. Date: May 27, 2004

(54) In vivo methods of determining activity of receptor-type kinase inhibitors

(76) Inventors: Kenneth A Thomas JR., Chatham Borough, NJ (US); Xianzhi Mao, Ambler, PA (US); Richard L Kendall, Thousand Oaks, CA (US)

Correspondence Address: MERCK AND CO INC, P O BOX 2000, RAHWAY, NJ, 070650907

(21) Appl. No.: 10/473,513

(22) PCT Filed: Mar. 29, 2002
(86) PCT No.: PCT/US02/09758

(51) Int. Cl. A61K 49/00; G01N 33/567

(52) U.S. Cl. 424/9.2; 435/7.21

(57) ABSTRACT

In vivo methods are disclosed for measuring compound inhibition of kinase receptor activity. Examples are provided which show a direct correlation between in vivo inhibition of KDR kinase inhibition and circulating blood and plasma levels of the inhibitor. These data are used to predict and validate non-quantifable in vitro measurements, such as murine endothelial cell IC₅₀ values. The in vivo potency of a compound determined by an assay of the present invention may be utilized to select dose amounts and frequencies for further preclinical animal model studies and human clinical studies designed to generate safety, potency and efficacy profiles for the respective inhibitor.

### CROSS-REFERENCE TO RELATED APPLICATIONS

This application claims benefit, under 35 U.S.C. §119(e), to U.S. provisional application 60/280,771 filed Apr. 2, 2001.

### STATEMENT REGARDING FEDERALLY-SPONSORED R&D

Not Applicable

### REFERENCE TO MICROFICHE APPENDIX

Not Applicable

### FIELD OF THE INVENTION

The present invention relates to in vivo methods of determining the ability of a compound to inhibit kinase receptor activity, including a receptor-type tyrosine kinase such as a mammalian KDR receptor, a member of the FLK family of receptor-type tyrosine kinases. These in vivo assays determine a correlation between kinase inhibitor activity and circulating plasma or blood levels of the inhibitor. In addition, calculation of the in vivo IC₅₀ of a specific compound utilizing this methodology in turn validates an arithmetic correlation between kinase receptor enzyme activity known for a first and second species and an in vitro IC₅₀ measurement known for the first species but not known for the second species, allowing for accurate derivation of the IC₅₀ for this second species. Such data as generated by the in vivo assays described herein are useful for formulation of protocols for subsequent preclinical animal studies as well as clinical human studies, both to test parameters which include but are not limited to safety, efficacy, dosing and formulation profiles for a potential kinase inhibitor.

### BACKGROUND OF THE INVENTION

Tyrosine kinases are a class of enzymes that catalyze the transfer of the terminal phosphate of adenosine triphosphate to tyrosine residues in protein substrates. Tyrosine kinases are believed, by way of substrate phosphorylation, to play critical roles in signal transduction for a number of cell functions and have been shown to be important contributing factors in cell proliferation, carcinogenesis and cell differentiation. Tyrosine kinases can be categorized as receptor type or non-receptor type. Receptor type tyrosine kinases typically have an extracellular, a transmembrane, and an intracellular portion, while non-receptor type tyrosine kinases typically are wholly intracellular, while examples exist of membrane receptors that upon ligand binding recruit intracellular kinases to bind to the intracellular portion of the receptor which, by itself, does not have kinase activity. The receptor-type tyrosine kinases are comprised of a large number of transmembrane receptors with diverse biological activity. In fact, about twenty different subfamilies of receptor-type tyrosine kinases have been identified. One tyrosine kinase subfamily, designated the HER subfamily, is comprised of EGFR, HER2, HER3, and HER4. Ligands of this subfamily of receptors include epithelial growth factor, TGF-α, amphiregulin, HB-EGF, betacellulin and heregulin. Another subfamily of these receptor-type tyrosine kinases is the insulin subfamily, which includes INS—R, IGF-IR, and IR-R. Then there is the FLK family which is comprised of the kinase insert domain receptor (KDR), the fms-like tyrosine kinase-1 (Flt-1), as well as the fms-like tyrosine kinase4 (Flt4). The FLK family of receptors is usually considered together with the PDGF receptor family due to the similarities of the two groups. For a detailed discussion of the receptor-type tyrosine kinases, see Plowman et al., 1994, DN&P 7(6): 334-339, which is hereby incorporated by reference.

The non-receptor type of tyrosine kinases is also comprised of numerous subfamilies, including Src, Frk, Btk, Csk, Abl, Zap70, Fes/Fps, Fak, Jak, Ack, and LIMK. Each of these subfamilies is further sub-divided into varying receptors. For example, the Src subfamily is one of the largest and includes Src, Yes, Fyn, Lyn, Lck, Blk, Hck, Fgr, and Yrk. The Src subfamily of enzymes has been linked to oncogenesis. For a more detailed discussion of the non-receptor type of tyrosine kinases, see Bolen, 1993, Oncogene, 8: 2025-2031, which is hereby incorporated by reference. Both receptor-type and non-receptor type tyrosine kinases are implicated in cellular signaling pathways leading to numerous pathogenic conditions, including cancer, psoriasis and hyperimmune responses.

The growth of blood vessels, or angiogenesis, is a normal embryonic and fetal developmental process. It appears to be driven principally by vascular endothelial growth factor (VEGF), a secreted protein that is chemotactic and mitogenic for vascular endothelial cells and can induce the cascade of events leading to vascular growth. Vascular endothelial cells form a luminal non-thrombogenic monolayer throughout the vascular system. Mitogens promote embryonic vascular development, growth, repair and angiogenesis mediated by these cells. Angiogenesis involves the proteolytic degradation of the basement membrane on which endothelial cells reside followed by the subsequent chemotactic migration and mitosis of these cells to support sustained growth of a new capillary shoot. One class of mitogens selective for vascular endothelial cells includes vascular endothelial growth factor (referred to as VEGF or VEGF-A) and the homologues placenta growth factor (PIGF), VEGF-B and VEGF-C. Homozygous and, surprisingly, heterozygous VEGF gene knockouts are embryonically lethal with diminished vascularization that is more extensive in the homozygous mice. The unusual lethal phenotype of the VEGF heterozygous gene knockout presumably reflects the importance of VEGF levels for normal embryonic development. However, in healthy adults the vasculature is stable with very little cellular turnover except for angiogenesis associated with tissue healing and the estrous cycle.

Aberrant angiogenesis is associated with several pathologies including neovascular ocular diseases, inflammation and a wide range of cancers. Regardless of which oncogene mutations lead to transformation, solid tumors require a vascular system to expand beyond small nodules limited by the diffusion of nutrients and metabolic by-products. Although tumor cells can initially colonize existing host capillaries, their growth leads to the collapse of these preexisting normal vessels resulting in hypoxia. Subsequent tumor growth requires neovascularization that is achieved by the ingrowth of new host blood vessels, denoted tumor angiogenesis. Tumors induce their neovascularization by secreting growth factors for vascular endothelial cells. The principal such factor that appears to support tumor angiogenesis is VEGF.

VEGF binds with high affinity to two transmembrane tyrosine kinase-linked receptors, Flt-1 (VEGFR-1) and KDR (Flk-1/VEGFR-2), that are expressed by vascular endothelial cells. In addition, Flt-1 is found on a variety of other types of cells including macrophages where it elicits a chemotactic but not a mitogenic response. Transfection experiments show that Flt-1 mediates neither substantial chemotaxis nor mitogenesis in vascular endothelial cells. Nevertheless, embryonically lethal homozygous mouse flt-1 gene knockouts exhibit vascular disorganization. An increased commitment to hemangioblast endothelial progenitor cell differentiation during very early embryonic development appears to alter vascular pattern formation leading to this disorganization. Flt-1 has also been implicated in the VEGF-mediated inhibition of antigen-presenting dendritic cell differentiation. The non-mitogenic role of Flt-1 in fully differentiated vascular endothelial cells is consistent with its binding to other homologous Flt-1-specific VEGF family members (PIGF, VEGF-B) that appear to be neither potent vascular endothelial cell mitogens nor angiogenic factors. VEGF-B, acting through Flt-1, has been reported to increase expression of urokinase and plasminogen activator inhibitor in vascular endothelial cells and metalloproteinases in smooth muscle cells. In contrast, transfection experiments demonstrate that KDR, which is selectively expressed by vascular endothelial cells and their progenitors, mediates an endothelial cell mitogenic and chemotactic response. In addition, embryonically lethal homozygous KDR gene knockout mice are essentially devoid of vascular endothelial cells and blood vessels.

Two viral proteins, HIV-1 tat and the orf virus-derived VEGF homolog VEGF-E, bind and activate KDR, are mitogenic for vascular endothelial cells and promote angiogenesis. Tat, but not VEGF-E, can also bind to Flt-1. Two other VEGF family members, VEGF-C and VEGF-D, selectively bind and activate Flt-4 (VEGFR-3), a receptor homolog primarily expressed on lymphatic endothelial cells, by which they can induce the growth of the lymphatic system, denoted lymphangiogenesis. However, upon proteolytic removal of long N- and C-terminal extensions observed to occur in vivo, VEGF-C and -D also acquire high affinity for KDR and become angiogenic vascular endothelial cell mitogens. Therefore, on the basis of receptor transfection and gene knockout experiments and the correlation among ligand binding, vascular endothelial cell mitogenesis and angiogenesis, the activation of the KDR receptor appears to be necessary and sufficient to trigger the VEGF-induced angiogenic cascade.

The binding of dimeric VEGF to the extracellular region of KDR promotes receptor dimerization that brings the intracellular tyrosine kinase domains together and promotes phosphorylation of several receptor tyrosine residues, at least some of which are critical for mitogenic signal transduction. Although often described as autophosphorylation, some or all of the tyrosine residues are probably transphosphorylated by the action of one tyrosine kinase on its dimeric partner. Phosphorylation of two tyrosine residues (1054 and 1059) on the “activation loop” near the catalytic site increases effective catalytic activity by decreasing the K_{M} values for ATP and peptide substrates with little, if any, effect on the intrinsic catalytic efficiency as reflected by k_{cat}. The mechanism of KDR enzymatic activation might be similar to what is thought to occur in other kinases in which, prior to phosphorylation of their tyrosine residues, the activation loops partially occlude the substrate binding regions. Autophosphorylation of the tyrosine residues within the activation loop alters its conformation thereby increasing access to the substrate binding sites. Phosphorylation of several other tyrosine residues, including those in the intracellular juxtamembrane region, the large “insert” loop and the C-terminal region, serve to generate binding sites for signal transduction proteins that assemble on KDR to form a functional activation complex. Once activated, KDR initiates a signal transduction cascade, is internalized and ultimately degraded. Inhibition of the VEGF/KDR system has been shown to inhibit VEGF-dependent tumor angiogenesis and growth in several animal models. As the mitogenically and angiogenically competent VEGF receptor, KDR is a particularly attractive target to antagonize VEGF-dependent tumor angiogenesis and growth.

Vascular growth in the retina leads to visual degeneration culminating in blindness. VEGF accounts for most of the angiogenic activity produced in or near the retina in diabetic retinopathy. Ocular VEGF mRNA and protein are elevated by conditions such as retinal vein occlusion in primates and decreased PO₂ levels in mice that lead to neovascularization. Intraocular injections of either anti-VEGF mono-clonal antibodies or VEGF receptor immunofusions inhibit ocular neovascularization in rodent and primate models. Regardless of the cause of induction of VEGF in human diabetic retinopathy, inhibition of ocular VEGF is useful in treating the disease.

Expression of VEGF is also significantly increased in hypoxic regions of animal and human tumors adjacent to areas of necrosis. Monoclonal and polyclonal anti-VEGF antibodies inhibit the growth of human tumors in nude mice. Although these same tumor cells continue to express VEGF in culture, the antibodies do not diminish the mitotic rate of most, if not all, tumor cells derived from cells other than vascular endothelial cells themselves. Thus tumor-derived VEGF does not function as an autocrine mitogenic factor for most tumors. Therefore, VEGF contributes to tumor growth in vivo by promoting angiogenesis through its paracrine vascular endothelial cell chemotactic and mitogenic activities. These monoclonal antibodies also inhibit the growth of typically less well vascularized human colon cancers in athymic mice and decrease the number of tumors arising from inoculated cells. Viral expression of a VEGF-binding construct of Flk-1, the mouse KDR receptor homologue, truncated to eliminate the cytoplasmic tyrosine kinase domains but retaining a membrane anchor, virtually abolishes the growth of a transplantable glioblastoma in mice presumably by the dominant negative mechanism of heterodimer formation with membrane-spanning endothelial cell VEGF receptors. Embryonic stem cells, which normally grow as solid tumors in nude mice, do not produce detectable tumors if both VEGF alleles are knocked out. Taken together, these data indicate the role of VEGF in the growth of solid tumors. Therefore, the angiogenically competent VEGF receptor KDR is implicated in pathological neoangiogenesis, and inhibitors of this receptor are useful in the treatment of diseases in which neoangiogenesis is part of the overall pathology, e.g., diabetic retinal vascularization, various forms of cancer as well as forms of inflammation such as rheumatoid arthritis, psoriasis, contact dermatitis and hypersensitivity reaction.

Mukhopadhyay et al., 1998, Cancer Res. 58: 1278-1284 shows stimulation of mesenteric KDR phosphorylation by i.p. injected VEGF.

Kasahara et al., 2000, J. Clin. Invest. 106: 1311-1319 shows inhibition of VEGF-induced phosphorylation by the Sugen KDR kinase inhibitor SU5416.

It will be advantageous to identify an in vivo-based assay which accurately determines the ability of a compound to inhibit receptor activity, such as the ability of a compound to inhibit VEGF-induced activity of KDR. The present invention addresses and meets this need by disclosing an assay which allows determination of KDR kinase inhibition as a function of the circulating plasma concentration of an inhibitor. The assays as disclosed herein allow for direct correlation of inhibition of KDR kinase activity with circulating plasma inhibitor levels, anti-angiogenic activity and the inhibition of tumor xenograft growth.

### SUMMARY OF THE INVENTION

The present invention relates to in vivo methods of determining the ability of a compound to inhibit kinase receptor activity, including but not limited to a receptor-type tyrosine kinase, a non-receptor-type tyrosine kinase and/or a serine/threonine receptor kinase. The methodology disclosed herein is particularly useful for determining the ability of a test compound or compounds to inhibit activity of a receptor-type tyrosine kinase. These assays allow for a direct in vivo correlation between the ability of a test compound to interact with a specific receptor or receptor type and the effect that receptor binding of the test compound has on a measurable biological or physiological event.

For example, a portion of the present invention relates to an in vivo assay measuring KDR kinase inhibition as a function of the circulating plasma concentration of an inhibitor. The vascular endothelial growth factor (VEGF) receptor KDR mediates the endothelial cell mitogenic and angiogenic activity of this growth factor. VEGF binding to KDR induces receptor dimerization. The intracellular tyrosine kinase domains are then activated by tyrosine phosphorylation, which increases their binding to substrates and complexation with downstream signal transduction proteins. This portion of the present invention therefore relates to an assay which monitors the inhibition in vivo of VEGF-induced KDR tyrosine phosphorylation by KDR kinase inhibitors. The assay allows for determination of in vivo IC₅₀ values by measuring the inhibition of KDR tyrosine autophosphorylation as a function of compound plasma concentration.

The in vivo potency of a compound determined by an assay of the present invention may be utilized to select dose amounts and frequencies for further preclinical animal model studies and/or human clinical studies designed to generate safety, potency and efficacy profiles for the respective inhibitor. For example, an exemplified portion of the present invention relates to measuring the IC₅₀ levels for various KDR inhibitors. The in vivo potency of a compound determined by this mouse KDR inhibition assay may be used to select dose amounts and frequencies for anti-angiogenesis and tumor xenograft growth inhibition efficacy studies.

An advantage of the assay disclosed herein is the determination of receptor kinase inhibition, including but not limited to KDR kinase inhibition, as a function of the circulating plasma concentration of an inhibitor. This allows for a direct correlation between inhibition of receptor activity and circulating plasma inhibitor levels, while also being useful in studying various dosing and frequency issues in respective animal models associated with the targeted disease or disorder.

Therefore, the present invention relates to an assay which determines the in vivo potency of kinase receptor inhibitor compounds, such as KDR kinase inhibitors. Inhibition can be directly related to local inhibitor concentrations including plasma and blood concentrations. Inhibition can be monitored as a function of dose levels, frequencies, routes of administration and time after dosing. The assays as exemplified herein for a VEGF/KDR-based assay relate further to additional assays. The exemplified assays may be adapted to monitor inhibitors of proteins other than KDR, including any receptor that may undergo chemical modification upon activation, such as other kinases such as a receptor-type tyrosine kinase, a non-receptor-type tyrosine kinase and/or a serine/threonine kinase receptor. The assay may also be adapted to multiple tissues in animal models, beyond the mouse lung tissue source disclosed herein, to include any tissue which contains an adequate amount of the receptor on or within various cell types of the tissue. The assay of the present invention may also be extended to additional mammalian species, including but not limited to rat, dog, rabbits, non-human primates and humans.

The present invention also relates to calculation of the in vivo IC₅₀ of a specific compound in a second species by utilizing an arithmetic correlation between kinase receptor enzyme activity known for the first species and a second species and an in vitro cellular potency IC₅₀ measurement known for the first species but not known for the second species; allowing for accurate derivation of the in vivo IC₅₀ for this second species, despite the possible absence of in vitro assay which might allow a prediction of the IC₅₀ for the second species. In other words, prior to the development of the methods disclosed herein, the skilled artisan would not have been comfortable utilizing such a calculation, based on in vitro data for three of a possible four variables. This disclosure shows excellent correlation with observed (i.e., directly measured, as in Example Section 1) in vivo IC₅₀ for a specific compound and the calculated value using the three of four variables to solve for the unknown variable (i.e., cell IC₅₀ for the second species, such as mouse). This showing instills a level of confidence in the artisan to, if necessary, bypass certain in vitro assays (e.g., when mouse EC cells are not readily available) but still be able to confidently predict the in vivo IC₅₀ in that same species (e.g., the IC₅₀ in a mouse model where mouse EC cells are not readily available for an in vitro assay).

Therefore, as exemplified herein, the present invention relates to assays which quantitatively measure the inhibition of a kinase receptor (such as KDR) in vivo, which allows for correlation of this data with inhibitory potency of the inhibitor on endothelial cells in vitro that is scaled to account for KDR species differences.

### BRIEF DESCRIPTION OF THE DRAWINGS

FIG. 1 shows the determination of in vivo IC₅₀ for compound #1 plotted as a function of the corresponding plasma concentration of compound #1 for each animal treated with inhibitor. KDR tyrosine phosphorylation of inhibitor-treated animals is then presented as a percentage of KDR phosphorylation in animals receiving the vehicle control which is set to 100%.

FIG. 2 shows the correlation among compounds 1-6 as assayed in the in vivo assay (described in Example Section 1) between the calculated and observed KDR tyrosine phosphorylation IC₅₀ values.

FIG. 3 shows, for compound #1, representative in vitro endothelial cell mitogen assay plots of VEGF-stimulated and unstimulated inhibitor dose-response assays.

### DETAILED DESCRIPTION OF THE INVENTION

The present invention relates to methods of monitoring the level of in vivo inhibition of ligand-induced activity (such as kinase phosphorylation) of a specific kinase receptor, including but not limited to a receptor-type tyrosine kinase, a non-receptor-type tyrosine kinase and/or a serine/threonine kinase. The methodology disclosed herein is particularly useful for determining the ability of a test compound or compounds to inhibit activity of a receptor-type tyrosine kinase. The invention is exemplified herein as an assay that monitors the inhibition of VEGF-induced tyrosine phosphorylation of murine lung KDR by a test KDR kinase inhibitor(s). This assay is utilized to obtain in vivo IC₅₀ values by measuring the inhibition of KDR tyrosine phosphorylation as a function of the plasma concentration of the test inhibitor(s). Thus, the present invention relates to an assay which determines the in vivo potency of a the test compound/inhibitor. This information is particularly useful for initial selection of dose amounts and frequency of administration for a particular test compound in safety assessment studies as well as determining dosing and frequency levels for human clinical trials for a particular test compound which possesses the ability to inhibit a respective kinase receptor. For example, it is shown herein that calculation of in vivo potency (i.e., IC₅₀ value) of a specific KDR kinase inhibitor can be used to determine dosing and frequency of the inhibitor compound for angiogenesis and tumor xenograft growth inhibition studies and well as being able to monitor the effects of inhibitors on tumor endothelial cell KDR phosphorylation and levels in mice. Therefore, the present invention fulfills a specific void in drug development protocols wherein no useful methodology was available to the artisan to monitor the in vivo KDR inhibition by KDR kinase inhibitors. It is shown herein, and exemplified utilizing a mouse lung KDR-based assay, that it is now possible to determine in vivo inhibition of KDR by KDR inhibitors as a function of the circulating plasma concentration of the KDR inhibitor. With these data, it is then possible to directly correlate inhibition of KDR kinase activity with plasma inhibitor levels, anti-angiogenic activity and inhibition of tumor xenograft growth.

As noted in the previous paragraph, it is contemplated that the present invention may be applied to various kinase receptors, including but not necessarily limited to a receptor-type tyrosine kinase, a non-receptor-type tyrosine kinase and/or a serine/threonine kinase. It will be within the purview of the artisan to adapt a specific kinase receptor chosen for study with a reasonable tissue(s) for harvest either by sacrifice or biopsy from the test animal. The artisan may, depending upon the kinase receptor under study, have several tissues from which to select for analysis subsequent to harvest or biopsy. The recovered tissue, as described herein, may then be analyzed to determine the amount of inhibitor binding as a correlate to blood plasma levels within that specific test animal. Thus, the comparison of receptor inhibition to various concentration of inhibitor in multiple animals will allow for the direct determination of the in vivo potency of a specific test inhibitor compound.

The present invention may be practiced with any number of animal models systems deemed appropriate for studying of the effect of kinase receptor inhibitors. Such animal models will allow for assays which determine the effect of kinase inhibitors on ligand/receptor interactions as well as biological responses associated with interference of ligand-receptor interaction. Examples of useful animal models include but are not limited to rodents such as mouse and rat, canine (dog), rabbit, guinea pig, and non-human primates (such as but not limited to rhesus monkeys, chimpanzees and baboons). It is also within the scope of this invention to apply the in vivo assay of the present invention to determine the IC₅₀ of a specific compound with a human subject. As an example, but certainly not a limitation, the assays of the present invention could be applied to human subjects wherein human tissue is removed (such as a bone marrow biopsy from a leukemic patient), other types of tumor biopsies, or possibly from a sample of peripheral mononuclear blood cells (PMBCs). It will then be possible to measure, ex vivo, either the inhibition of a specific kinase receptor (such as KDR) or, in the alternative, measure a more abundant, homologous kinase receptor (such as Flt-1, Flt4, c-kit, c-fms or Flt-3, when compared to the target KDR receptor, as well as the PDGFR-α and PDGFR-β receptors) for which a direct comparison of compound inhibition may be made. The latter strategy allows for the inference of the potency on, for example, KDR, on the basis of its potency on one of these other, more abundant receptors. As noted above, in the case of kinase targets such as bone marrow cells and PMBCs, it is envisioned that the patient would be dosed with the respective inhibitor, followed by removal of this tissue from the patient and the assay completed ex vivo, as to avoid direct administration of the ligand to a human patient.

An inhibitor compound for use in this assay may be any compound which may potentially have therapeutic activity in mammals, especially for eventual human administration of the respective inhibitor. Types of inhibitor compounds include but are not necessarily limited to non-proteinaceous organic or inorganic molecules, peptides, proteins, nucleic acid molecules such as DNA or RNA (and especially single stranded antisense molecules which may inhibit kinase receptor binding and/or activation). One such nucleic acid or corresponding expressed protein or portion thereof comprises a soluble form of Flt-1 or KDR, including but not limited to the forms disclosed in U.S. Pat. Nos. 5,712,380 and 5,861,484, which are hereby incorporated by reference. A preferred soluble version of a receptor-type kinase from the FLK family includes the sFLT-1 protein as disclosed in SEQ ID NO:6 of the '380 and '484 patents. The assay as exemplified herein utilizes various small organic molecules which have previously been shown as KDR kinase inhibitors.

The vascular endothelial growth factor (VEGF) receptor KDR mediates the endothelial cell mitogenic and angiogenic activity of this growth factor. VEGF binding to KDR induces receptor dimerization. The intracellular tyrosine kinase domains are then activated by tyrosine phosphorylated which increases their binding to substrates and complexation with downstream signal transduction proteins. This assay monitors the inhibition of VEGF-induced tyrosine phosphorylation of mouse lung KDR by KDR kinase inhibitors. A particular embodiment of the present invention relates to a method of determining the in vivo KDR kinase inhibition by an administered test inhibitor compound as a function of the circulating plasma concentration of that inhibitor. It is used to obtain in vivo IC₅₀ values by measuring the inhibition of KDR tyrosine autophosphorylation as a function of compound plasma concentration. The in vivo potency of a compound determined by this assay is used to select dose amounts and frequencies for angiogenesis and tumor xenograft growth inhibition efficacy studies. It also has been adapted to monitor the effect of inhibitors on tumor endothelial cell KDR phosphorylation and levels in mice.

The present invention is exemplified herein by calculating the IC₅₀ for various known KDR inhibitors in a mouse study model. Again, this particular exemplification of the present invention, as with any other chosen receptor, can be studied in any other useful animal model. In terms of in vivo assays to determine IC₅₀ values for KDR kinase inhibitors, especially useful animal models are mouse, rat and dog. Any particular KDR inhibitor, such as compounds 1-6 as shown in Example Section 1, may be administered to mice by known enteral or parenteral routes, including but not limited to oral administration (such as oral gavage, sublingual administration or rectal administration), injection directly into the blood stream (such as intravenous or intra-arterial administration), or various parenteral routes (such as intraperitoneal and subcutaneous routes such as intramuscular), respiratory-based administration via an aerosol, and administration under the skin (i.e., transdermal, transcutaneous or percutaneous), as well as topical administration of the formulated compound of interest. Compounds are typically administered at several dose levels to Nu/Nu female mice. A preferred form of administration in mouse studies of KDR kinase receptor activity are via oral gavage or intraperitoneal administration. After various times, typically between 1 and 24 hr after dosing, the tyrosine autophosphorylation of KDR receptors is stimulated by a tail-vein injection of VEGF (including various forms of human VEGF, such as humanVEGF₁₆₅, as well as other forms of mammalian VEGF, including but not limited to rat VEGF₁₆₄ five minutes before sacrifice. Blood samples are taken to determine compound concentrations in plasma by LCMSMS. The lungs, containing the first major capillary bed encountered by agents injected into the venous system and initially determined by Western blots to be one of the tissues with higher levels of KDR, are removed and quickly frozen and stored in liquid nitrogen until processed. The frozen tissue is weighed then pulverized in liquid nitrogen and a lysis buffer is added to the tissue, followed by incubation and centrifugation. The cleared supernatant is immunoprecipitated by an anti-KDR antibody. Immunoprecipitated KDR-antibody complexes are captured and fractionated by SDS-polyacrylamide gel electrophoresis. The fractionated immunocomplexes are blotted onto an appropriate membrane, probed with anti-phosphotyrosine antibody. The anti-phosphotyrosine antibody is detected and quantified by known methodology. The blot is then stripped, re-probed with the anti-KDR antibody and the KDR bands are again detected and quantified. The ratio of phosphorylated KDR/total KDR signals are calculated and expressed as a percent of VEGF-stimulated vehicle control-treated mice. The IC₅₀ is calculated by curve fitting relative KDR tyrosine phosphorylation as a function of plasma compound concentration. It is shown herein that within the set of several KDR kinase inhibitors for which mouse lung KDR tyrosine phosphorylation IC₅₀ values have been determined, there is a good correlation between this measure of in vivo potency and the IC₅₀ value in a cultured human endothelial cell mitogenesis assay multiplied by the ratio of the in vitro enzyme IC₅₀ values of mouse/human KDR kinase. This scaled value estimates the IC₅₀ for mouse endothelial cells which are not currently readily available as either pure cultures or stable cell lines to assay in culture. The calculated mouse endothelial cell IC₅₀ is calculated as (avg. human endothelial cell IC₅₀)×(avg. mouse KDR IC₅₀/avg. human KDR IC₅₀). The correlation between calculated and observed mouse lung IC₅₀ KDR kinase phosphorylation values is observed among numerous KDR inhibitor compounds, as shown in Example Section 1. Therefore, such an assay is used to determine the in vivo potency of a kinase inhibitor (such as one or more of compounds 1-6, disclosed herein) on a specific kinase receptor (KDR) following additional of the receptor ligand (mammalian VEGF). Inhibition can be (1) directly related to local inhibitor concentrations including plasma and blood concentrations; and, (2) monitored as a function of dose levels, frequencies, routes of administration and time after dosing. Therefore, general applications of the assay as exemplified herein may be adapted to assay inhibitors of other proteins that undergo chemical modification upon activation including other kinases noted herein. It can also be adapted to tissues other than lung and other species including humans.

The assays of the present invention thus allow for measurement of inhibitor potency via dose-response of compounds to inhibit kinase receptor phosphorylation in vivo (again, such as but not limited to KDR kinase phosphorylation). This data is especially useful in correlating the observed in vivo IC₅₀ from the respective species (e.g., such as mouse, rat and dog) with the in vitro human endothelial cell IC₅₀ scaled by the ratio of the IC₅₀ for the respective species/human KDR enzyme IC₅₀. In other words, the assay confirms and reinforces, and therefore the present invention further relates to, a calculation of the predicted endothelial cell IC₅₀ for a species where such in vitro cell populations are not readily available, such as a murine system. The determination of the endothelial cell IC₅₀ for insertion into this arithmetic equation may be generated by any method known in the art to effectively measure KDR kinase activity (and the inhibition thereof). Such an assay may involve the well known endothelial cell mitogen assay (as shown in Example Section 2), or other related assays which directly measure KDR kinase phosphorylation, including but not limited to measurement of in vitro autophosphorylation of KDR in endothelial cells. In such methodology, primary human umbilical vein endothelial cells (HUVEC) are incubated in the presence of the inhibitor prior to activation of KDR by addition of VEGF. Cell lysates are recovered and subjected to analysis to determine the amount inhibitor binding. Another example is the in vitro measurement of KDR kinase inhibition in a cell line (preferably a cell line which does not normally express KDR, such as HEK 293 cells) transfected with a species specific KDR gene, or kinase relevant portion thereof. Such an assay ultimately depends on a quantitative analysis of the effect of the test compound to inhibit KDR phosphyorylation. In other words, the various assays for determining the in vitro IC₅₀ for endothelial cell mitogenesis or KDR kinase activity are interchangeable; predicted to quantify similar IC₅₀ concentrations. This is so since the binding of dimeric VEGF ligand by the extracellular recognition site on KDR dimerizes the receptor bringing the intracellular kinase domains into proximity where they can tyrosine phosphorylate each other. Phosphorylation of tyrosine residues on the activation loop of KDR effectively increases the catalytic activity of the enzyme at subsaturating substrate concentrations by increasing the enzyme affinity (i.e. lowered Kms) for ATP and peptide substrate (Kendall, et al., 1999, J. Biol. Chem. 274: 6453-6460). Activated KDR also phosphorylates other KDR tyrosine residues that serve as docking sites for downstream signal transduction proteins thereby initiating the cascade of events culminating in mitosis. VEGF-induced mitogenesis of vascular endothelial cells, monitored by DNA synthesis, is a direct consequence of VEGF-induced tyrosine phosphorylation of KDR; the inhibition of KDR tyrosine phosphorylation in cells is also directly related to the inhibition of its induction of DNA synthesis. Therefore, in VEGF-stimulated vascular endothelial cells comparable KDR kinase inhibitor potencies (IC₅₀s) can be determined by dose-response assays either of the inhibition of the initial event of KDR tyrosine phosphorylation or of the resulting DNA synthesis. Because of the low numbers of KDR receptors per endothelial cell, it is preferable to measure the inhibition of endothelial cell DNA synthesis since it has a higher signal-to-noise than is observed for the inhibition of KDR kinase tyrosine phosphorylation for the limited number of cells on the small surface area of each well within the 96 well plates used for routine high throughput dose-response assays.

Therefore, the present invention relates to an in vivo method of determining inhibition of a specific kinase receptor activity as a function of the circulating concentration of a test compound in a non-human subject which comprises (a) administering the test compound to a test subject, the test subject being a non-human mammal, (b) stimulating the kinase receptor activity within the test subject, (c) collecting a blood or plasma sample from the test subject, (d) determining the concentration of the test compound from the sample of step c, (e) collecting a tissue or blood cell sample from the test subject that contains a measurable amount of the kinase receptor, (f) determining the relative proportion of the receptor that is phosphorylated within the sample compared to a samples that were not dosed with the test compound of step e); and, (g) correlating the effect of the test compound to inhibit the kinase receptor phosphorylation as a function of the blood or plasma level of the test compound as determined in step c).

The present invention also relates to an in vivo method of determining the IC₅₀ for a test compound in relation to a specific kinase receptor for a non-human subject which comprises (a) administering multiple doses of differing concentration of the test compound to multiple test subjects of the same mammalian species, excluding a human, wherein a single test subject receives a single dose of the test compound, (b) stimulating the kinase receptor activity within each of the test subjects, (c) collecting a blood or plasma sample from each of the test subjects, (d) determining the concentration of the test compound from the sample of step c), (e) collecting a tissue sample from each test subject wherein the tissue sample contains a measurable amount of the kinase receptor and is from a similar source for each test subject, (f) determining the relative ratio of phosphorylated to non-phosphorylated kinase receptor within each sample, compared to a placebo control, of step e), and (g) correlating kinase receptor phosphorylation of step e) for each subject as a function of the blood or plasma level of the test compound as determined in step c), resulting in an in vivo observed IC₅₀ value for the test compound.

In view of the ability to generate in vivo KDR phosphorylation-based values of inhibitor activity, the present invention further relates to a method of predicting the in vivo IC₅₀ for a test compound in a second species in relation to a specific kinase receptor protein, which comprises (a) measuring the in vitro enzymatic IC₅₀ for a first species and the second species of the specific kinase receptor protein, (b) measuring the in vitro cellular response IC₅₀ or kinase receptor IC₅₀ of the first species, (c) multiplying the ratio of the first species in vitro enzymatic IC₅₀ to the second species in vitro enzymatic IC₅₀ of step a) by the in vitro cellular response IC₅₀ or kinase receptor IC₅₀ measurement of step b), which results in a calculation of the predicted in vivo cellular response or kinase receptor IC₅₀ of the second species for a test compound targeting the specific kinase receptor protein.

The above methods are especially useful for test subjects selected from the group consisting of the genus Mus, Rattus and Canis, the kinase receptor is a member of the FLK receptor family, as well as additional kinases such as PDGFR-α and PDGFR-β. An exemplified and prefrered FLK receptor is KDR and a preferred tissue type is lung tissue from a test subject of the genus Mus.

The following non-limiting Examples are presented to better illustrate the invention.

### EXAMPLE 1

Mouse KDR Autophosphorylation

Material and Methods—The structure of compounds tested in this exemplification of the present invention are as follows:

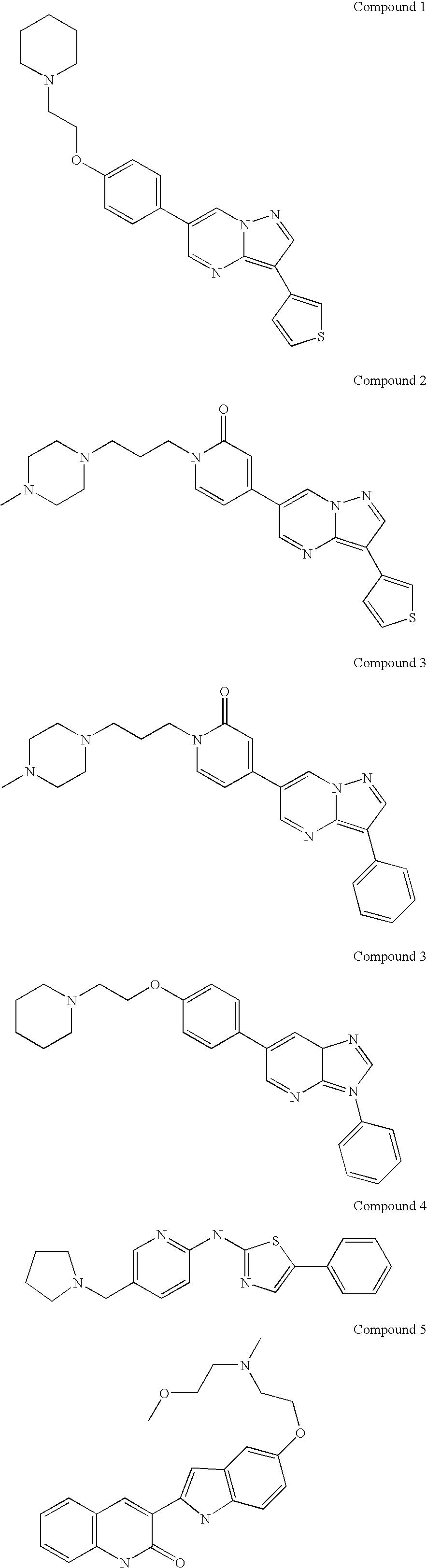

Compounds are typically administered at several dose levels either by intraperitoneal injection or by oral gavage to Nu/Nu female nude mice. After various times, typically between 1 and 24 hr after dosing, the tyrosine autophosphorylation of KDR receptors is stimulated by a tail-vein injection of human VEGF₁₆₅ five minutes before sacrifice. Blood samples are taken to determine compound concentrations in plasma by liquid chromatography mass spectroscopy mass spectroscopy (LCMSMS). The lungs, containing the first major capillary bed encountered by agents injected into the venous system and initially determined by Western blots to be one of the tissues with higher levels of KDR, are removed and quickly frozen and stored in liquid nitrogen until processed. The frozen tissue is weighed then pulverized in liquid nitrogen. Lysis buffer (20 mM HEPES, pH 7.5, 150 mM NaCl, 1% Triton X-100, 300 mM pervanadate, 50 mM NaF, 1 mM Microcystin-LR with proteinase inhibitor cocktail) is added to the tissue for 2 hr at 4° C. The lysate is then centrifuged at 14,000 rpm for 10 min at 4° C. and the pre-cleared supernatant is immunoprecipitated by an anti-KDR antibody [SC-504, SantaCruz Biotechnology]. Immunoprecipitated KDR-antibody complexes are captured by Protein A-Sepharose CL 4B beads and fractionated by sodium dodecyl sulfate-polyacrylamide gel electrophoresis. The fractionated immunocomplexes are blotted onto a PVDF membrane, probed with anti-phosphotyrosine antibody [#05-321, Upstate Biotechnology], detected by enzyme-linked chemiluminescence [RPN 2109, Amersham Pharmacia Biotech.] and quantified using a Molecular Dynamics densitometer. The immunoblot is then stripped, re-probed with anti-KDR antibody [SC-6251, Santa Cruz Biotechnology] and the KDR bands are detected and quantified as above. The ratio of phosphorylated KDR/total KDR signals are calculated and expressed as a percent of VEGF-stimulated, vehicle control-treated mice. The IC₅₀ is calculated by curve fitting relative KDR tyrosine phosphorylation as a function of plasma compound concentration.

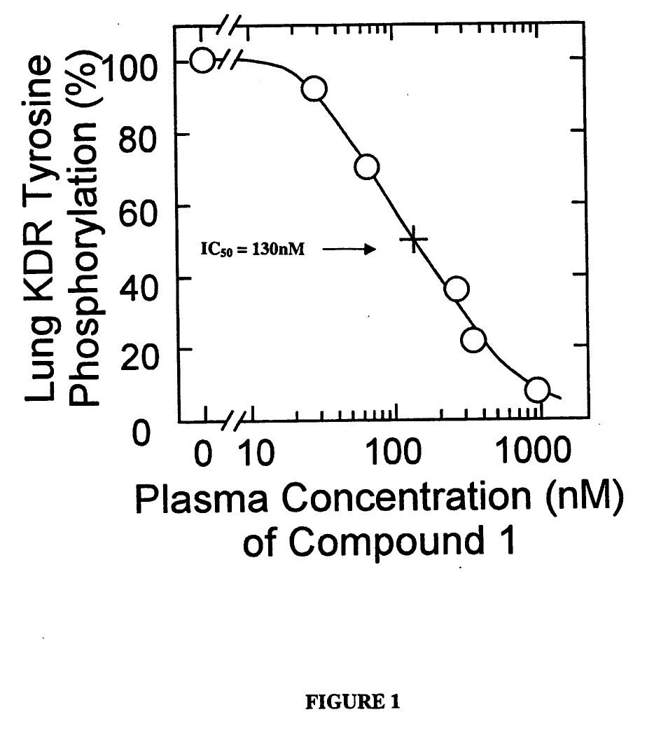

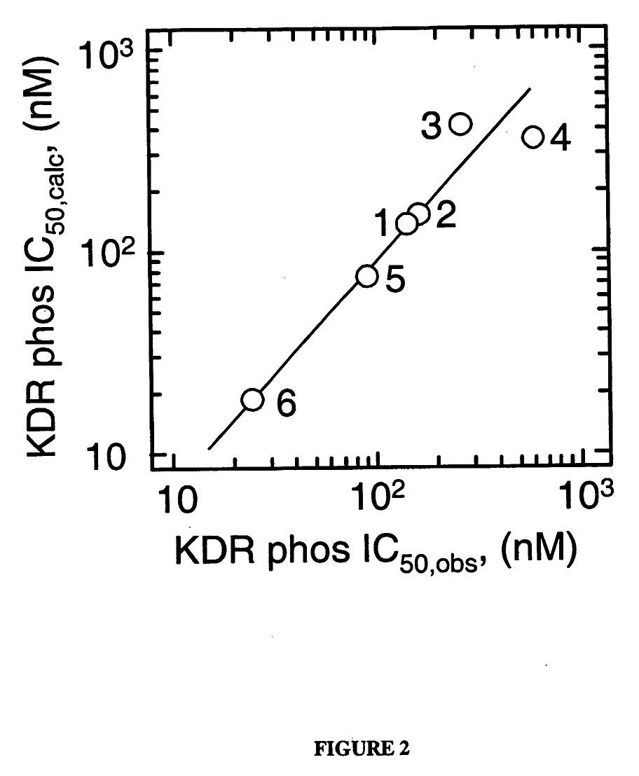

Results—Compound #1 inhibits VEGF-stimulated mouse lung KDR autophosphorylation in a dose dependent manner with an IC₅₀ value of 130 nM as shown in FIG. 1. Within the set of several KDR kinase inhibitors for which IC₅₀ values have been determined, there is a good correlation between this measure of in vivo potency and the IC₅₀ value in the cultured human endothelial cell mitogenesis assay (ECMA) multiplied by the ratio of in vitro enzyme IC₅₀ values of mouse/human KDR kinase. This scaled ECMA value estimates the IC₅₀ for mouse endothelial cells which are not available as either pure cultures or stable cell lines to assay in culture. For compound 1, the calculated mouse endothelial cell IC₅₀ is =(avg. ECMA)×(avg. mouse KDR IC₅₀/avg. human KDR IC₅₀)=18.0 nM×(24 nM/3.3 nM)=130 nM. As shown in FIG. 2, there is a good correlation between the calculated and the observed IC₅₀ values within the set of compounds, representing several different core structures, that have been evaluated in FIG. 2. No additional correction for protein binding, apart from that which is intrinsic to the ECMA-cell based assay, is required to achieve this correlation. This value is in good agreement with the 130 nM IC₅₀ calculated by scaling the human endothelial cell mitogenesis IC₅₀ by the ratio of the mouse/human KDR kinase enzyme inhibition IC₅₀ values. The implication of the good agreement between the observed and calculated IC₅₀ values over the set of compounds assayed in this example is that the in vivo KDR phosphorylation IC₅₀ values in other species may also be calculated by a similar scaling algorithm using the IC₅₀ values for the corresponding KDR kinase enzyme activities. Therefore, in humans the IC₅₀ value of compound #1 for endothelial cells directly exposed to circulating plasma should correspond to the range encompassed by an in vitro human endothelial cell mitogenesis assay and/or an assays that directly measure KDR phosphorylation in either cultured human vascular endothelial cells or a cultured human embryonic kidney (HEK293) cell line stably transfected to express KDR.

### EXAMPLE 2

In Vitro Human Endothelial Cell Mitogenesis Assay (ECMA)

Methods—Early passage human vascular endothelial cells (HUVECs) are seeded in 96 well tissue culture plates at a density of 3.5×10³ cells per well in 0.1 ml of assay medium (DMEM plus 10% fetal calf serum). Cells are growth arrested at 37° C. in a humidified atmosphere containing 5% CO₂. After 24 hrs, medium is replaced with 0.1 ml of fresh assay medium containing either vehicle or test compound. Each dilution of test and control compounds and vehicle controls are assayed in triplicate. Test compounds are dissolved and serially diluted in 100% DMSO to keep them soluble through the dilution series. Inhibitors at each concentration are then diluted 400-fold in assay media (final 0.25% concentration of DMSO) of which 0.1 ml is used to replace the spent media in each well. A parallel dose-response assay is done with a potent compound standard used in sequential week assays as an internal positive control. After a 2 hr preincubation with compound, cells are fully stimulated with either 50 ng/ml of VEGF. Assay media alone is added to an unstimulated control group. After 24 hrs [³H]thymidine is added to a final concentration of 0.8 μCi/well. Following incubation for an additional 72 hrs, assay media is removed, cells are washed, trypsinized and collected on 96 well filtration plates. Scintillation cocktail is added to each well and cell associated radioactivity is determined in a MicroBeta Liquid Scintillation Counter. The mean cpm values are determined for each set of triplicate wells and corrected by subtracting mean background counts from wells containing HUVECs that were treated 0.25% DMSO vehicle control medium but with neither VEGF, nor inhibitor. Compound inhibition of VEGF—induced DNA synthesis is expressed as the percent of the fully stimulated minus unstimulated responses.

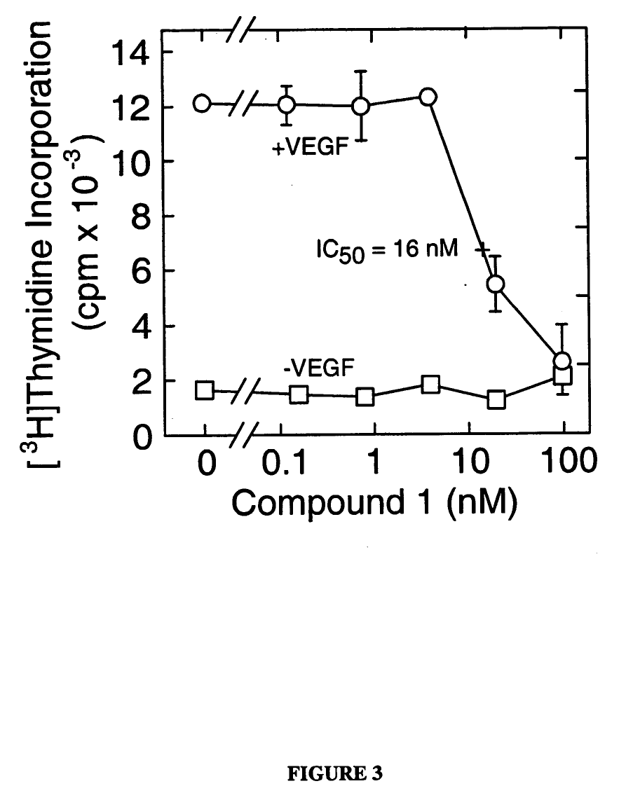

Results—A dose-response assay of compound #1 in VEGF— stimulated and unstimulated HUVECs is shown in FIG. 3. FIG. 3 shows the response plotted as [³H]thymidine incorporation as a function of the concentration of compound #1. Compound #1 was assayed five times as a function of dose in ECMA to give a mean IC₅₀ value of 18.0 nM.

The present invention is not to be limited in scope by the specific embodiments described herein. Indeed, various modifications of the invention in addition to those described herein will become apparent to those skilled in the art from the foregoing description. Such modifications may be related to the method of calculating in vitro IC₅₀ values, as discussed herein. These potential modifications are intended to fall within the scope of the appended claims.

What is claimed is:

1. An in vivo method of determining inhibition of a specific kinase receptor activity as a function of the circulating concentration of a test compound, which comprises:
a) administering the test compound to a test subject, the test subject being a non-human mammal;
b) stimulating the kinase receptor activity within the test subject;
c) collecting a blood or plasma sample from the test subject;
d) determining the concentration of the test compound from the sample of step c;
e) collecting a tissue or blood cell sample from the test subject that contains a measurable amount of the kinase receptor;
f) determining the relative proportion of the receptor that is phosphorylated within the sample compared to a samples that were not dosed with the test compound of step e); and,
g) correlating the effect of the test compound to inhibit the kinase receptor phosphorylation as a function of the blood or plasma level of the test compound as determined in step c).

2. The method of claim 1 wherein the test subject is selected from the group consisting of the genus Mus, Rattus and Canis.

3. The method of claim 1 wherein the kinase receptor is a member of the FLK receptor family.

4. The method of claim 3 wherein the FLK receptor is KDR.

5. The method of claim 4 wherein the test subject is selected from the genus Mus.

6. The method of claim 5 wherein the tissue sample is lung tissue.

7. An in vivo method of determining the IC₅₀ for a test compound in relation to a specific kinase receptor, which comprises:
a) administering multiple doses of differing concentration of the test compound to multiple test subjects of the same mammalian species, excluding a human, wherein a single test subject receives a single dose of the test compound;
b) stimulating the kinase receptor activity within each of the test subjects;
c) collecting a blood or plasma sample from each of the test subjects;
d) determining the concentration of the test compound from the sample of step c);
e) collecting a tissue sample from each test subject wherein the tissue sample contains a measurable amount of the kinase receptor and is from a similar source for each test subject;
f) determining the relative ratio of phosphorylated to non-phosphorylated kinase receptor within each sample, compared to a placebo control, of step e); and,
g) correlating kinase receptor phosphorylation of step e) for each subject as a function of the blood or plasma level of the test compound as determined in step c), resulting in an in vivo observed IC₅₀ value for the test compound.

8. The method of claim 7 wherein the test subject is selected from the group consisting of the genus Mus, Rattus and Canis.

9. The method of claim 7 wherein the kinase receptor is a member of the FLK receptor family.

10. The method of claim 9 wherein the FLK receptor is KDR.

11. The method of claim 10 wherein the test subject is selected from the genus Mus.

12. The method of claim 11 wherein the tissue sample is lung tissue.

13. A method of predicting the in vivo IC₅₀ for a test compound in a second species in relation to a specific kinase receptor protein, which comprises:
a) measuring the in vitro enzymatic IC₅₀ for a first species and the second species of the specific kinase receptor protein;
b) measuring the in vitro cellular response IC₅₀ or kinase receptor IC₅₀ of the first species;
c) multiplying the ratio of the first species in vitro enzymatic IC₅₀ to the second species in vitro enzymatic IC₅₀ of step a) by the in vitro cellular response IC₅₀ or kinase receptor IC₅₀ measurement of step b),
resulting in calculation of the predicted in vivo cellular response or kinase receptor IC₅₀ of the second species for a test compound targeting the specific kinase receptor protein.

14. The method of claim 13 wherein the kinase receptor is a member of the FLK receptor family.

15. The method of claim 14 wherein the FLK receptor is KDR.

16. The method of claim 15 wherein the first species is human.

17. The method of claim 16 wherein the second species is from the genus Mus.
